(19) United States
(12) Patent Application Publication
(10) Pub. No.: US 2005/0158285 A1
(43) Pub. Date: Jul. 21, 2005

(54) Method of re-profiling adult stem cells using embryonic stem cell electromagnetic signals

(76) Inventor: Vincent C. Giampapa, Little Falls, NJ (US)

Correspondence Address: MELVIN K. SILVERMAN, 500 WEST CYPRESS CREEK ROAD, SUITE 500, FT. LAUDERDALE, FL, 33309, US

(21) Appl. No.: 11/008,374

(22) Filed: Dec. 9, 2004

Related U.S. Application Data
(60) Provisional application No. 60/528,264, filed on Dec. 9, 2003.

(51) Int. Cl. C12N 5/08; C12N 15/00

(52) U.S. Cl. 424/93.7; 435/440; 435/366

(57) ABSTRACT

Methods of re-profiling adult stem cells are disclosed, which include recording electromagnetic signals from an embryonic stem cell, cytoplasm thereof, individual components of the cytoplasm, nucleus thereof, or chromosome from the nucleus, using an electronic device and storing the electromagnetic signals; and then transmitting the electromagnetic signals to the adult stem cell of a mammal the same species in vitro, and transferring transmitted adult stem cell back into the mammal. Alternatively, the recorded electromagnetic signals are transmitted to the adult stem cells in vivo. The methods also include reactivating silenced genes in the mammal prior to transmitting the electromagnetic signals to the adult stem cell of the mammal. Transmission of electromagnetic signals from embryonic stem cells to adult stem cells provides molecular information of embryonic environment, which induces aged adult stem cells functioning as young cells.

### CROSS REFERENCE TO RELATED APPLICATIONS

This application claims the benefit under 35 USC 119 (e) of the provisional patent application Ser. No. 60/528,264, filed on Dec. 9, 2003, which is herein incorporated by reference in its entirety.

### FIELD OF THE INVENTION

The present invention relates to the method of reprofiling adult stem cells using embryonic stem cell electromagnetic signals.

### BACKGROUND OF THE INVENTION

Over the past decade, embryonic stem cells have been shown to hold a promise for regeneration of tissues in adults. Embryonic stem cells possess the capacity to restore not only tissues in general, but selective embryonic cell lines including ectodermal, endodermal and mesodermal derived tissues.

The term stem cells is used to describe those cells that serve as a normal reservoir for new cells needed to replace damaged or dying cells. Stem cells are broadly divided into four groups: adult stem cells, fetal stem cells, embryonic stem cells, and nuclear transplant stem cells.

After the formation of stem cells in the fetus, some tissues and organs continue to maintain a population of stem cells throughout childhood and into adulthood. Even though these cells appear early in development, they are called adult stem cells. For example, blood vessels readily repair damage and can actually regenerate. That is the bone marrow which maintains a population of blood cell stem cells and produces billions of new blood cells each day. Cells that line the stomach and intestines are also regularly replaced. Another example is spermatogenesis. The fetal testes is populated with stem cells that are not actually stimulated to produce more cells until puberty, but once sperm production is initiated, it continues throughout the life of the male. Billions of new sperm are produced daily. As the fundamental features of adult stem cells, they maintain the ability to divide throughout life and give rise to specific cell types.

However, there is a limit to the number of times mammalian cells can divide. As this limit is approached, the cells age, a person or a mammal ages. This mortality has been defined at the DNA level. The sequence TTAGGG is found at the ends of human chromosomes, the chromosomal region known as the telomere. TTAGGG repeats hundreds of times effectively capping the chromosome end. As the cell ages, with each round of cell division some of the TTAGGG telomere units are lost, continually shortening the chromosomes. At some point late in life so many telomeric units have been lost that the cell senesces, or goes into a resting stage. Therefore, it has been found that with aging, both the numbers of adult stem cells and the functional capacity thereof markedly deceased. Hence, it is believed that if a sufficient amount of youthful adult stem cells could be maintained in the human body, RNA transcription, which initiates the replication of the DNA molecule, could be maintained at a sufficient level to offset the effects of aging, otherwise caused by failures in the cell replication process.

On the other hand, it has been found that the aging process also involves silencing of the genes through methylation of promoter sequences and the deacetylation of histones. Most genes are active during initial embryonal development, then they begin to be blocked through methylation and deacetylation. However, this trend accelerates especially after age 25, which increasing numbers of genes silenced as a person grows older. When this silencing affects tumor suppressor genes, an aging person may develop cancer. Two biochemical processes play an important part in gene silencing: deacetylation of the histones in short-term silencing and methylation of promoter sequences of the genes in long-term silencing, the later is a major factor leading to progressive aging and ultimately, death.

In past 35 years, Burzynski and colleagues at Burzynski Clinic, Houston, have isolated a number of substances and formulated 12 pharmaceutical agents with the ability to reactivate tumor suppressor genes in cancer patients through inhibition of methylation and deacetylation, these being named molecular switches antineoplastons (Burzynski S. R. Gene Silencing—a new theory of aging, Medical Hypotheses, 2003, 60(4), 578-583; Burzynski S. R. Potential of antineoplastons in diseases of old age, Drugs Aging 1995, 7, 157-167; Burzynski S. R. Novel differentiation inducers, In D. Adeam (ed). Recent Advances in Chemotherapy, Munich, Puturamed Publisher, 1992; U.S. Pat. No. 4,918,193 (to Burzynski S. R.), entitled “Purified antineoplastons fractions and method of treating neoplastic diseases”). It has been found that these antineoplastons, at the same time they reactivate tumor suppressor gene, also turn on other genes which can provide aging patients with a number of benefits, such as increased energy, increased epidermization and nail growth, reduction of wrinkles and hyperpigmentation spots, reduction of cholesterol and triglyceride concentration in blood, improvement in autoimmune diseases, Parkinson's disease, and in benign prostate hypertrophy, and effect on DNA including protection against carcinogen, activation of tumor suppressor genes p53 and p21, interruption of signal transmission in ras oncogene pathway, demethylation of promoter sequences of tumor suppressor genes, inhibition of deacetylation of histones.

It is known that among 3000 genes in average human chromosome only 300 are selected for activation at any given time. Inactive genes remain coiled and are not transcribed and wrapped around the histone core in nucleosomes. Each core histone possesses a tail protruding outside the nucleosome and containing lysine which is a positively charged amino acid. It was found that lysine residues in the histone tails are selectively modified through acetylation and methylation. A positive charge of lysine can be neutralized by acetylation, weakening the interaction of the tail with DNA, preventing nucleosomes form folding into stable fibers and helping gene expression. Currently, sodium phenylbutyrate is one of the best known histone deacetylation inhibiting agent. Another inhibitor is depsipeptide, also known as FK228, which is in Phase I clinical trials. A novel class of inhibitors, sulfonamide anilides have also been described (Fournel M. et al, Sulfonamide anilides, a novel class of histones deacetylase inhibitors, are antiproliferative against human tumors, Cancer Res 2002, 62: 4625-4630).

Methylation of gene promoter sequences converts cytosine inside DNA into methylcytosine by addition of methyl group at the 5 position of the pyrimidine ring. Under normal conditions in the buman body, only cytosine followed by guanosine is methylated (CpG nuclelotides). CpG islands are frequently located in promoter sequences of the genes. Hypermethylation of these islands is associated with vrioius types of cancers. Excessive methylation of CpG islands in promoter regions will silence genes (Ordway J. M. et al. Cell Growth Differ 2002, 13: 149-162).

Cytoplasm is the viscid, semifluid matter contained within the plasma membrane of a cell. The aqueous component of the cytoplasm is the cytosol, which includes ions and soluble macromolecules, such as proteins, RNA, carbohydrates and enzymes. The insoluble constituents of the cytoplasm include the organelles and the cytoskeleton. While all cells possess a cytoplasm, cells from the different biological domains can differ widely in the characteristics of their cytoplasm. Various cytoplasm components are involved in cell division process, for example growth factors and cell cycle regulating molecules.

The quantitative structure-activity relationship theory of molecular signaling believes that two structurally matching molecular objects exchange specific information to induce cellular function by physical contact between a ligand 2 and a receptor 4, as illustrated in FIG. 1. However, recent molecular signaling research has revealed that the electromagnetic signals emitted by the agonist molecule can activate specific receptor of the agonist molecule and induce cellular functions.

The ability of low frequency electromagnetic (EM) signals to stimulate particular cell receptors through co-resonance to thus activate and induce specific cell functions has been studied for many years by Benveniste and colleagues. Benveniste et al reported that they had activated various biological system using electromagnetic signals in a range of KHz of an agonist molecule, instead of using the molecule itself, and that the target cell respond to such signals, applied electronically thru water to target cells, similarly to the response to the agonist molecule.

More specifically, Thomas. et al., FASEB J. 1995, 9:A227, reported that the molecular signal of 4-phorbol-12-b-myristate-13-acetate (PMA) can be electronically transmitted and modulate neutrophils reactive oxygen metabolite production as if PMA itself was added to the cells. The source tube containing 1 um of PMA was placed on the input coil at room temperature, and target cells (1 million per ml) on the output coil at 37C in an incubator. After transmitting the PMA signals through an amplifier for 15 minutes, an increase in reactive oxygen metabolite production of the neutrophils was observed immediately.

Furthermore, Benvensiste et al have digitally recorded the electromagnetic signals of the agonist molecules and then replayed to the target cells, or organs. Benvensiste et al, “Specificity of the Digitized Molecular Signal” Experimental Biology '98 (FASEB), San Francisco, Apr. 20, 1998, have used a purpose-designed transducer and a sound card-equipped computer to digitize, record and replay the signals of acetylcholine and histamine, in the range of 0-44 kHz, to isolated perfused guinea pig hearts. The results have shown that digitized EM signals of acetylcholine and histamine influenced cardiac function of guinea pig in the same way as the original molecules did. This indicates that the molecular signal is composed of waveforms in the 0-44 kHz range which are specific to each molecular entity. Therefore, the electromagnetic signals of molecules can be digitized, stored, and transmitted digitally to the target cells or organs.

Moreover, given that essentially all biochemical reactions occur in aqueous media, the electrical properties of water itself are believed to play a signification role in EM-induced bio-effects in signaling and binding processes, as shown by Aissa et al, “Molecular Signalling of High Dilution or by Means of Electronic Circuitry” J. Immunol. 150: 146A and Aissa, et al (1994), “Transfer of the Molecular Signal by Electrical Amplification” FASEB J. 8: A398 (Abstract).

Furthermore, the effects of low energy electromagnetic fields on cell signal transduction and upon gene-specific molecules have been reported. For example, the enzyme known as DNA-dependent RNA polymerase, is affected by an EM frequency (72 Hz) pulsed magnetic field. Goodman et al, “Electronic Magnetic Fields Induce Cellular Transcription” Science 220: 128 (1983), reported increased gene transcription in dipteran salivary gland cells, compared to unexposed control cells, as assessed by transcription audio radiography and cytological nick translation. Phillips et al, “Effect of 72 Hz Pulsed Magnetic Field Exposure on Macromolecular Synthesis in CCRS-CEM cells”, Cancer Biochem. Biophysical. 12: 1 (1991), reported increased incorporation of uridine into both total cellular RNA and mRNA in T-cells exposed to a 72 Hz pulsed magnetic field. It is also noted that magnetic field responsive genes have been identified by Wu, et al; 45 Chinese Science Bulletin 1, p. 1006-1010, June, 2000, as has the effect of magnetic fields in protein DNA binding, Lin, et al. 1:J. Cell BioChem 1998, 70(3): 297-303.

### SUMMARY OF THE INVENTION

In one embodiment, the present invention is directed to a method for re-profiling adult stem cells, which comprises the steps of: collecting an amount of cytoplasm from embryonic stem cells of a mammal; recording electromagnetic signals from said cytoplasm using an electronic device and storing said electromagnetic signals; obtaining an adult stem cell from an adult mammal of a same species, and placing said adult stem cell in an aqueous medium; transmitting said electromagnetic signals to said adult stem cell; and transferring transmitted adult stem cell back into said adult mammal, wherein the transmitted adult stem cell is re-profiled to function as a young cell. The recorded electromagnetic signals can be digitized and transmitted in the digital form. The electromagnetic signals are in a frequency range from 0 Hz to about 500 kHz.

The method further comprises replicating transmitted adult stem cell in a culture medium for a number of cell cycles without differentiation, and transferring said transmitted adult stem cell and replicated adult stem cells into said adult mammal. During the cell replication process the recorded electromagnetic signals can be further transmitted to the adult stem cells. Alternatively, the recorded electromagnetic signals are transmitted to the adult stem cells in vivo, by a direct transmission to a part of the body of a subject, without harvesting the adult stem cells from the subject.

Furthermore, the method of the present invention can include recording electromagnetic signals from individual components of embryonic stem cell cytoplasm, which are involved in the stem cell division process, and then transmitting the recorded electromagnetic signals to adult stem cells in vitro or in vivo.

In a further embodiment, the method of the present invention includes recording electromagnetic signals from an embryonic stem cell nucleus, a chromosome, or chromosomes of the nucleus in an aqueous medium, and then transmitting the recorded electromagnetic signals to adult stem cells in vitro or in vivo. Further, the aqueous medium can also include cytoplasm of the embryonic stem cell.

In another embodiment, the method of the present invention includes recording electromagnetic signals from a whole embryonic stem cell, which includes molecular information from nucleus, cytoplasm and the cellular membrane receptors, and then transmitting the recorded electromagnetic signals to adult stem cells in vitro or in vivo.

In yet a further embodiment, the method of the present invention further includes a step of administering to the mammal an inhibitor of methylation of DNA, or an inhibitor of histone deacetylation to reactivate silenced genes prior to obtaining the adult stem cell from the mammal. Therefore, the method enables the reactivated genes to receive and respond to the transmitted electromagnetic signals from the embryonic stem cell.

### BRIEF DESCRIPTION OF THE DRAWINGS

FIG. 1 is a representational view of the key-to-keyhole model of molecular interaction.

FIG. 2 is a representational view showing electromagnetic (EM) signals functioning as a biological equivalent to an agonist molecule.

FIG. 3 is a representational view showing the electromagnetic signals reaching the cell nucleus.

### DETAILED DESCRIPTION OF THE INVENTION

In one embodiment, the present invention provides a method of re-profiling adult stem cells. The method comprises the steps of: collecting an amount of cytoplasm from embryonic stem cells of a mammal; recording electromagnetic signals from the cytoplasm using an electronic device and storing the electromagnetic signals; obtaining an adult stem cell from an adult mammal and placing the adult stem cell in an aqueous medium; transmitting the electromagnetic signals to the adult stem cell; and transferring the transmitted adult stem cell back into the adult mammal. Furthermore, the recorded electromagnetic signals can be digitized, and transmitted digitally.

It is noted that the term “embryonic stem cell” used herein denotes undifferentiated cell derived from the inner cell mass of a blastocyst that will give rise to every cell of the adult organism. The embryonic stem cell cytoplasm can be obtained from isolated embryonic stem cells, or from the embryonic stem cells cultured in laboratory.

The term “adult stem cell” used herein denotes the stem cell of a mammal's organ, such as bone marrow, liver, lining of gut and spermatozoa. The term “aged adult stem cell” used herein denotes the adult stem cell obtained from an adult human having an age about 30 year old or older, or equivalent thereof for other mammals. In the same sense, the term “young adult stem cell” used herein denotes the adult stem cell obtained from an adult human having an age less than 30 year old, or equivalent thereof for other mammals.

Collecting embryonic stem cell cytoplasm can be achieved using a syringe or a micropipette such as that used currently in the nuclear transfer. The methods and processes of obtaining and isolating adult stem cells from a mammalian organ, and transferring the adult stem cells back into the mammalian body are known to those skilled in the art, and can be utilized for the purpose of the present invention.

In general, the electromagnetic signals to be recorded are in a frequency range from 0 Hz to about 500 kHz, and preferably less than 100 kHz. The electronic devices known in the art, suitable for recording the electromagnetic signals in this frequency range can be used for the purpose of the present invention. More specifically, the devices and processes developed and utilized by Benveniste and colleagues at Digital Biology Laboratory, Clamart, France, can be used for the purpose of the present invention. To facilitate the recording process, the embryonic cytoplasm can be diluted with an aqueous medium, such as deionized water, or deuterium depleted water. The transmitting process is carried out in an aqueous medium, such as in a saline solution.

As earlier noted above, aged adult stem cells have stored within them genomic profiles from both early and embryonic life. However, due to external processes, such as shortening of chromosomal telomeres, methylation of stem cell specific DNA promoter sequence and deacetylation of histones, the some genes become silenced, thereby inhibiting the capacity of aged adult stem cells to function in a youthful fashion.

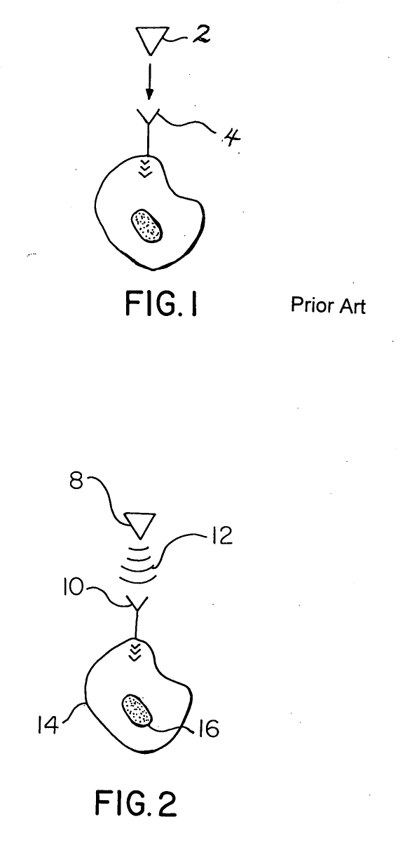

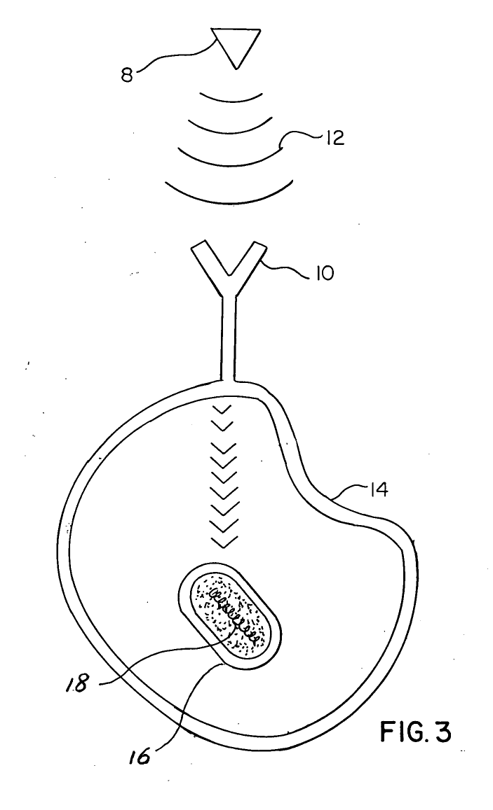

As illustrated schematically in FIGS. 2 and 3, the electromagnetic signals 12 recorded from the embryonic stem cell cytoplasm 8, or individual components of the cytoplasm, nucleus or chromosome from the nucleus, of the embryonic stem cell, as described hereinafter, are transmitted to the adult stem cells 14. The cell membrane receptor 10, cell nucleus 16 of the adult stem cells 14 and DNA 18 of cell nucleus 16 receive molecular information from embryonic environment, which induces the adult stem cell to function as a young cell and avoids silencing or resting due to aging. Consequently, aged adult stem cells so treated are re-profiled to function as young or less aged cells.

The phrase “re-profiling aged adult stem cell” used herein denotes the results achieved by inducing an aged adult stem cell to function as a young stem cell by transmitting to the aged adult stem cell of the electromagnetic signals from an embryonic stem cell, embryonic stem cell cytoplasm or components thereof, such as reducing methylation of DNA segments, reducing DNA damage occurring during cellular aging, such as shortening of telomere, and enhancing DNA repair processes.

In a further embodiment, the method of the present invention comprises the steps of isolating one or more individual components from the embryonic stem cell cytoplasm and placing the individual components in an aqueous medium; recording electromagnetic signals from the individual components in the aqueous medium using an electronic device and storing the electromagnetic signals; and then transmitting the electromagnetic signals to the adult stem cell from an adult mammal of the same species and transferring transmitted adult stem cell back into the adult mammal, as described above.

Suitable individual components of the cytoplasm include, but are not limited to, mRNA, antisense RNA, complimentary RNA, micro RNA and Ribo RNA, growth factors, maturation promotion factor (MPF), enzymes such as telomerase, DNA polymerase, RNA polymerase, DNA ligase, DNA endonuclease, cyclins A, D1, D2, D3, E B, and cyclin dependent kinases (cdk), such as cdk2 (cdc2), cdk4, cdk5 and cdk1. These individual components are known involved in the process of cell division. The methods and processes of isolating various individual components are known to those skilled in the art, and can be utilized for the purpose of the present invention.

Transmitting the electromagnetic signals from the individual component, individually or in combination thereof, provides specific molecular information from embryonic environment to induce the aged adult stem cell to function as a young cell.

In another embodiment, the method of the present invention comprises the steps of obtaining a mammalian embryonic stem cell nucleus, and placing the embryonic stem cell nucleus in an aqueous medium; recording electromagnetic signals from the embryonic stem cell nucleus in the aqueous medium using an electronic device and storing the electromagnetic signals; and then transmitting the electromagnetic signals to the adult stem cell from an adult mammal of the same species and transferring transmitted adult stem cell back into the adult mammal, as described above. Furthermore, the aqueous medium suspending the embryonic stem cell nucleus can further contain the cytoplasm of the embryonic stem cell. The methods and processes of isolating stem cell nucleus are known to those skilled in the art, such as those used in nuclear transfer, and can be utilized for the purpose of the present invention.

Furthermore, in this embodiment the method can further comprise the step of obtaining individual chromosome or chromosomes from the nucleus of the embryonic stem cell, and the electromagnetic signals are recorded from individual chromosome or chromosomes in the aqueous medium.

In this case, the recorded electromagnetic signals include molecular information of the embryonic stem cell nucleus, individual chromosome or chromosomes of the nucleus, which upon transmitting to the adult stem cell activates nuclear functions and induces the aged adult stem cell to function as a young cell. Moreover, in the presence of cytoplasm in the aqueous medium, the recorded electromagnetic signals include molecular information from both the nucleus and the cytoplasm of the embryonic stem cell, which resemble an intracellular environment of the embryonic stem cell.

In yet a further embodiment, the method of the present invention comprises recording electromagnetic signals from whole embryonic stem cells in an aqueous medium using an electronic device and storing the electromagnetic signals, and then transmitting the electromagnetic signals to the adult stem cell from an adult mammal of the same species and transferring transmitted adult stem cell back into the adult mammal, as described above.

To record the electromagnetic signals from whole embryonic stem cells, the embryonic stem cells need to be preserved in an isotonic aqueous solution, such as a saline solution. The background signals from the saline solution can be subtracted electronically from the recorded signals if desired. The electromagnetic signals from whole embryonic stem cells include the information from the nucleus, cytoplasm and the cellular membrane receptors.

Furthermore, the electromagnetic signals can also be recorded from ectodermal, endodermal, or mesodermal stem cell lines, either from the cytoplasm, individual components thereof, or the whole stem cells, and then transmitted to specific adult stem cells of interest, in various manners described above, to stimulate tissue repairing for a specific or derived cell line, in order to repair cell damage due to aging or diseases.

In a further embodiment, the method of the present invention can include a step of reactivating silenced genes of the aged adult stem cell prior to transmission of the embryonic stem cell electromagnetic signals to the aged adult stem cell. As discussed previously in the Background of the Invention, reactivation of silenced genes can be achieved through inhibition of methylation of DNA and histone deacetylation using antineoplastons from Burzynski et al. and other DNA methylation and histone deacetylation inhibitors known in the art. Suitable examples of DNA methylation inhibitors and histone deacetylation inhibitors include, but are not limited to, antineoplastons AS2-1 and AS5, which are synthetic derivatives of glutamine and phenylacetate, activing the p53 gene (Burzynski S. R. Potential of antineoplastons in diseases of old age, Drugs Aging 1995, 7, 157-167); antineoplastons A2, A3 and A5, which inhibit methylation of DNA through destabilization of the complex of methionine adenosyltransferase (MAT), methyltransferase (MT) and S-adenosylmomocyteine hydrolase (SAHH) (Liau M. C. et al, Altered methylation complex isozyme as selective targets for cancer chemotherapy, Drugs Exp Clin Res 1986; 12 Suppl. 1: 313-326); sodium phenylbutyrate (U.S. Pat. No. 6,372,938), depsipeptide (FK228), sulfonamide anilides (Fournel M. et al, Sulfonamide anilides, a novel class of histones deacetylase inhibitors, are antiproliferative against human tumors, Cancer Res 2002, 62: 4625-4630), and demethylation agents such as cytidine analogs including 5-azacytidine and 5-aza-2′-deoxycytidine. All references cited herein are incorporated by reference in their entirety.

The antineoplaston, or other DNA methylation and histone deacetylation inhibitors can be administered orally by the donor of the adult stem cell, following the dosage and duration of administration known in the art, prior to obtaining the adult stem cell. Through inhibiting methylation of DNA, particularly promoter gene sequence, and deacetylation of histones to reactivate silenced genes in the adult stem cells, the reactivated genes in the harvested adult stem cells can also receive and response to the electromagnetic signals from the embryonic stem cells.

Preferably, the method of the present invention further comprise replicating transmitted adult stem cell in a culture medium for a number of cell division cycles in order to increase the numbers of adult stem cells without differentiation, and transferring the transmitted adult stem cell and replicated adult stem cells into the adult mammal. Preferably, during the replication process, the recorded electromagnetic signals are also transmitted to the adult stem cells. During the culturing, cellular function and viability can be monitored. Culturing stem cell in vitro without having differentiation occurring is known in the art, more particularly the Replicell technology developed by Aastrom Bioscience, Inc., Ann Arbor, Mich.

Alternatively, to increase the number of adult stem cells prior to obtaining them from the body, medications which are known to increase the production of stem cells can be administered systematically. For example, Neupogen® (manufactured by Amgen, Inc.) known to stimulate production and release of adult stem cells in the bone marrow, can be administered prior to harvesting the adult stem cells from the bone marrow.

For the transmitted and replicated adult stem cells, the length of telomeres can be determined using Southern blot and flow cytometric analyses on the control cell which is treated using the same process and conditions, but not to be transferred back to the mammalian body. The increase of telomere length of the transmitted and replicated adult stem cells indicates the reverse of cellular aging, and restoring aged adult stem cells to a youthful state. Gene expression can also be analyzed before and after treating the adult stem cells as described above by using DNA microarrays (also referred to as gene chips) to identify the genes which are transcription active or inactive.

It should be understood for the purpose of the present invention that the embryonic stem cells and the adult stem cells are obtained from the same species, for example, collecting embryonic stem cell cytoplasm from a human embryo, and obtaining adult stem cells from an adult human. Furthermore, the donor of the adult stem cells is preferably the recipient of the transmitted and replicated adult stem cells, therefore, there is no concern of tissue rejection.

In an additional embodiment, the method of the present invention can include transmitting the recorded electromagnetic signals to adult stem cells in a part of the body of an adult mammal of the same species, such as to bone marrow, or liver. In this manner, the adult stem cells are not isolated from the body, and the signals are directly transmitted through the tissue, wherein a large quantity of water in the body functions as the transmission medium.

Furthermore, the medications, such as Neupogen®, can be administered to a subject first to stimulate production of adult stem cells in the bone marrow, and then the recorded electromagnetic signals can be transmitted to the bone marrow, to enhance the efficiency of the method.

While there has been shown and described the preferred embodiment of the instant invention it is to be appreciated that the invention may be embodied otherwise than is herein specifically shown and described and that, within said embodiment, certain changes may be made in the form and arrangement of the parts without departing from the underlying ideas or principles of this invention as set forth in the claims appended herewith.

1. A method of re-profiling adult stem cells comprising the steps of:
(a) collecting an amount of cytoplasm from a mammalian embryonic stem cell;
(b) recording electromagnetic signals from said cytoplasm using an electronic device and storing said electromagnetic signals;
(c) obtaining an adult stem cell from a mammal of a same species, and placing said adult stem cell in an aqueous medium;
(d) transmitting said electromagnetic signals to said adult stem cell; and
(e) transferring transmitted adult stem cell from step (d) back into said mammal.

2. The method of claim 1 further comprising digitizing said electromagnetic signals after said recording.

3. The method of claim 1, wherein said electromagnetic signals are in a frequency range from 0 Hz to about 500 kHz.

4. The method of claim 1 further comprising administering to said mammal an inhibitor of methylation of DNA to reactivate silenced genes prior to obtaining said adult stem cell from said mammal.

5. The method of claim 4, wherein said inhibitor of methylation of DNA is at least one selected from the group consisting of antineoplaston, 5-azacytidine and 5-aza-2′-deoxycytidine.

6. The method of claim 1 further comprising administering to said mammal an inhibitor of histone deacetylation to reactivate silenced genes prior to obtaining said adult stem cell from said mammal.

7. The method of claim 6, wherein said inhibitor of histone deacetylation is at least one selected from the group consisting of antineoplaston, phenylbutyrate, depsipeptide (FK228), and sulfonamide anilide.

8. The method of claim 1 further comprising replicating transmitted adult stem cell from step (d) in a culture medium for a number of cell cycles without differentiation, then transferring said transmitted adult stem cell and replicated adult stem cells into said mammal.

9. The method of claim 8 further comprising transmitting said electromagnetic signals to said transmitted adult stem cell during cell replication.

11. The method of claim 1 further comprising isolating an individual component from said cytoplasm and placing said individual component in a second aqueous medium, wherein said recording electromagnetic signals from said cytoplasm is recording electromagnetic signals from said individual component in said second aqueous medium.

12. The method of claim 11, wherein said individual component is at least one selected from the group consisting of mRNA, antisense RNA, complimentary RNA, micro RNA and Ribo RNA, growth factor, maturation promotion factor, telomerase, DNA polymerase, RNA polymerase, DNA ligase, DNA endonuclease, cyclins including cyclin A, D1, D2, D3, E and B, and cyclin dependent kinase including cdk2 (cdc2), cdk4, cdk5 and cdk1.

13. A method of re-profiling adult stem cells comprising the steps of:
(a) obtaining a mammalian embryonic stem cell nucleus and placing said embryonic stem cell nucleus in a first aqueous medium;
(b) recording electromagnetic signals from said embryonic stem cell nucleus in said first aqueous medium using an electronic device and storing said electromagnetic signals;
(c) obtaining an adult stem cell from a mammal of a same species, and placing said adult stem cell in a second aqueous medium;
(d) transmitting said electromagnetic signals to said adult stem cell; and
(e) transferring transmitted adult stem cell from step (d) back into said mammal.

14. The method of claim 13 further comprising digitizing said electromagnetic signals after said recording.

15. The method of claim 13, wherein said electromagnetic signals are in a frequency range from 0 Hz to about 500 kHz.

16. The method of claim 13 further comprising administering to said mammal an inhibitor of methylation of DNA to reactivate silenced genes prior to obtaining said adult stem cell from said mammal.

17. The method of claim 16, wherein said inhibitor of methylation of DNA is at least one selected from the group consisting of antineoplaston, 5-azacytidine and 5-aza-2′-deoxycytidine.

18. The method of claim 13 further comprising administering to said mammal an inhibitor of histone deacetylation to reactivate silenced genes prior to obtaining said adult stem cell from said mammal.

19. The method of claim 18, wherein said inhibitor of histone deacetylation is at least one selected from the group consisting of antineoplaston, phenylbutyrate, depsipeptide (FK228), and sulfonamide anilide.

20. The method of claim 13 further comprising obtaining chromosome for said embryonic stem cell nucleus and placing said chromosome in a second aqueous medium, wherein said recording electromagnetic signals from said embryonic stem cell nucleus is recording electromagnetic signals from said chromosome in said second aqueous medium.

21. The method of claim 13 further comprising replicating transmitted adult stem cell from step (d) in a culture medium for a number of cell cycles without differentiation, then transferring said transmitted adult stem cell and replicated adult stem cells into said mammal.

22. The method of claim 21 further comprising transmitting said electromagnetic signals to said transmitted adult stem cell during cell replication.

23. The method of claim 13, wherein said first aqueous medium further comprising cytoplasm of said embryonic stem cell.

24. A method of re-profiling adult stem cells comprising the steps of:
(a) collecting a cellular component from a mammalian embryonic stem cell;
(b) recording electromagnetic signals from said cellular component using an electronic device and storing said electromagnetic signals; and
(c) transmitting said electromagnetic signals to adult stem cells in a part of the body of a mammal of a same species.

25. The method of claim 24, wherein said cellular component is cytoplasm, an individual component of said cytoplasm, nucleus, or chromosome of said embryonic stem cell.

26. The method of claim 24 further comprising digitizing said electromagnetic signals after said recording.

27. The method of claim 24, wherein said electromagnetic signals are in a frequency range from 0 Hz to about 500 kHz.

28. The method of claim 24 further comprising administering to said mammal an inhibitor of methylation of DNA to reactivate silenced genes prior to transmitting said electromagnetic signals.

29. The method of claim 28, wherein said inhibitor of methylation of DNA is at least one selected from the group consisting of antineoplaston, 5-azacytidine and 5-aza-2′-deoxycytidine.

30. The method of claim 24 further comprising administering to said mammal an inhibitor of histone deacetylation to reactivate silenced genes prior to transmitting said electromagnetic signals.

31. The method of claim 30, wherein said inhibitor of histone deacetylation is at least one selected from the group consisting of antineoplaston, phenylbutyrate, depsipeptide (FK228), and sulfonamide anilide.

32. A method of re-profiling adult stem cells comprising the steps of:
(a) placing a mammalian embryonic stem cell in a first aqueous medium;
(b) recording electromagnetic signals from said embryonic stem cell using an electronic device and storing said electromagnetic signals;
(c) obtaining an adult stem cell from a mammal of a same species and placing said adult stem cell in a second aqueous medium;
(d) transmitting said electromagnetic signals to said adult stem cell; and
(e) transferring transmitted adult stem cell from step (d) back into said mammal.

33. The method of claim 32 further comprising digitizing said electromagnetic signals after said recording.

34. The method of claim 32, wherein said electromagnetic signals are in a frequency range from 0 Hz to about 500 kHz.

35. The method of claim 32 further comprising administering to said mammal an inhibitor of methylation of DNA to reactivate silenced genes prior to obtaining said adult stem cell from said mammal.

36. The method of claim 33, wherein said inhibitor of methylation of DNA is at least one selected from the group consisting of antineoplaston, 5-azacytidine and 5-aza-2′-deoxycytidine.

37. The method of claim 32 further comprising administering to said mammal an inhibitor of histone deacetylation to reactivate silenced genes prior to obtaining said adult stem cell from said mammal.

38. The method of claim 33, wherein said inhibitor of histone deacetylation is at least one selected from the group consisting of antineoplaston, phenylbutyrate, depsipeptide (FK228), and sulfonamide anilide.

39. The method of claim 32 further comprising replicating transmitted adult stem cell from step (d) in a culture medium for a number of cell cycles without differentiation, and transferring said transmitted adult stem cell and replicated adult stem cells into said mammal.
